(19) United States
(12) Patent Application Publication
(10) Pub. No.: US 2024/0238612 A1
(43) Pub. Date: Jul. 18, 2024

(54) METHOD FOR INTERVAL CLOSED LOOP ADAPTIVE TRANSCRANIAL PHOTOBIOMODULATION

(71) Applicant: SENS.AI INC., WHISTLER (CA)

(72) Inventors: Paola Telfer, Whistler (CA); Corey Julihn, Langley (CA)

(21) Appl. No.: 18/559,328

(22) PCT Filed: May 6, 2022
(86) PCT No.: PCT/IB2022/054240

§ 371 Date: Nov. 6, 2023

Related U.S. Application Data
(60) Provisional application No. 63/185,234, filed on May 6, 2021.

(51) Int. Cl. A61N 5/06

(52) U.S. Cl. CPC A61N 5/0622; A61N 2005/0626; A61N 2005/0647; A61N 2005/0651

(57) ABSTRACT

Methods for brainwave modulation using interval based closed-loop adaptive transcranial photobiomodulation are provided. The inventive methods utilize biometric sensors including Electroencephalogram (EEG) sensors to measure the biometric signals from a body and processes said signals to adapt patterned pulses of light in order to alter the electrical activity of the brain. Systems in the form of wearable devices such as headsets for carrying out the inventive methods are also provided.

### PRIOR RELATED APPLICATIONS

This application claims the benefit of priority of prior-filed U.S. Provisional Application 63/185,234.

### FIELD OF THE INVENTION

The present invention relates to methods for brainwave modulation using interval based closed-loop adaptive transcranial photobiomodulation. Specifically, the invention pertains to a method for using biometric sensors including Electroencephalogram (EEG) sensors to measure the biometric signals from a body and processing said signals to adapt patterned pulses of light in order to trigger photochemical changes within cellular structures and influence the electrical activity of the brain.

### BACKGROUND OF THE INVENTION

Influencing biometric signals from the body crosses many disciplines and methods including medicine, therapy, meditation, breathing exercises, biofeedback, neurofeedback and biostimulation. Neurostimulation is one form of biostimulation which involves the purposeful modulation of nervous system activity. One such method of neurostimulation known as Photobiomodulation (PBM) uses modulating near-infrared light to stimulate the nervous system.

Photobiomodulation is a form of infrared light therapy. Infrared light therapy can have positive effects on the skin, metabolic processes, the nervous system and immune system. It has been shown to increase collagen production for healthier skin.

Photobiomodulation techniques can stimulate the mitochondria in cells through the transfer of energy. Inside mitochondria, cytochrome oxidase has the ability to absorb red and near infrared light and convert it into energy—adenosine triphosphate (ADT). Transcranial photobiomodulation systems often transmit light at a wavelength between 633 and 810 nanometers with 810 nm being an ideal wavelength due to its ability to penetrate further into biological tissue.

Further, transcranial photobiomodulation is a neurotechnology technique used to modulate or alter an individual's brain activity creating a perceptible change in mental state which can be seen through changes in the electrical activity of the brain. Brainwave states can be defined as the collective electrical activity of a brain over a period of time; which can then be classified into a mental state such as tired, focused, stressed, creative, etc.

Other forms of brain stimulation include but are not limited to:
tACS—Transcranial Alternating Current Stimulation
tRNS—Transcranial Random Noise Stimulation
tDCS—Transcranial Direct Current Stimulation

To date, transcranial photobiomodulation has been deployed using either static lights or constant frequency light pulses. More recently, researchers have begun experimenting with varying the frequencies of the lights within a stimulation session. Using different pulse frequencies has been shown to result in different measured and subjectively felt effects reported by the subject. The varying effects of pulsed light stimulation are not yet fully understood. However, the inventors propose this effect is a direct result of varying the time brain cells are exposed to the light energy. Therefore these effects are a result of both frequency and duty cycle of the lights.

Existing methods have shown significant variations in results across different people, and even across the same person over time—sometimes resulting in decreased brainwave activity and other times resulting in increased brainwave activity. Some people have also reported headaches, nausea, dizziness and lightheadedness from existing tPBM techniques.

Light-based neurostimulation has been shown to differ significantly from electrical based stimulation; techniques used in existing electrical stimulation methods do not directly transfer to this field. Several studies have shown Photobiomodulation to have a biphasic dose-response curve known as the Arndt-Schulz Law (FIG. 4). Wherein low doses beyond a threshold have no effect and higher doses result in bio-inhibition of the transfer of energy. It has also been shown that the light penetration into biological tissue will vary from person to person based on differences in skin and skull thickness, as well as hair. Significantly, where Transcranial Photobiomodulation (tPBM) is concerned, in real world environments, measuring dose per centimeter squared (J/cm2) is not a direct measurement of energy absorption nor a measure of dosage for the individual person. Prior art also lacks an understanding of how changes to the dose over time influence the Arndt-Schulz biphasic response curve. Prior art devices apply a consistent energy dose output to all subjects. These devices do not account for differences in energy penetration and absorption.

Some devices used have been reported to cause headaches, nausea, dizziness and lightheadedness. Exceeding the dose absorption limit results in a drop in EEG power and reported side effects are a likely result of exceeding the dose absorption limit for the individual person.

The electrical activity of the brain is extremely complex. This can vary significantly from person to person and moment to moment, and at different locations within the brain. Importantly the electrical activity of a brain, or brainwaves, can be considered a non-stationary signal. Prior art has demonstrated that tPBM techniques can alter brainwave activity; however these attempts fail to account for the non-stationary nature of the brain.

### SUMMARY OF THE INVENTION

The current invention demonstrates significant improvements over prior art which lack the real-time, close-loop feedback mechanism used by current invention to adapt to different people during a session, the same person across multiple sessions and the non-stationary nature of brainwaves. Known approaches are further insufficient as they either employ static lights or lack the complex pulsed interval patterns defined by this method. Said patterns are critical for personalizing the energy over time independently of the LED duty cycle and pulse frequency. Furthermore, the present invention is able to measure and account for individualized rates of energy absorption and also resolves the issues in prior art related to headaches, nausea, dizziness and lightheadedness.

Results of tests conducted by the inventors and evidenced in FIG. 2 and FIG. 3 clearly show the present invention ability to increase targeted brainwave activity wherein methods used in prior art often result in no meaningful change or even response inhibition from the pre-stimulation baseline or as shown in FIG. 3 result in a significant decrease in targeted brainwave power.

The present invention pertains to a method for using biometric sensors including 1 or more Electroencephalogram (EEG) sensors to measure the biometric signals from a body and processing said signals to adapt pulses of light wherein light pulses are used to alter the electrical activity of the brain. Biometric sensors and photobiomodulation lights are placed on a body and said sensors are used to establish a baseline of biometric signals over a period of time. Further, the present invention measures the effect of the photobiomodulation and makes adjustments to said stimulation in an automated fashion, creating a closed-loop system.

The inventive method provides improvement over known methods which do not account for the non-stationary nature of brainwave activity nor adapt to differences between individuals nor the same individual over time.

### BRIEF DESCRIPTION OF THE DRAWINGS

FIG. 1 shows a wearable headset and connected mobile device and computer system in accordance with an embodiment of the present invention.

FIG. 2 is a chart showing EEG readings before and after constant frequency transcranial photobiomodulation stimulation on a person.

FIG. 3 is a chart showing EEG readings before and after interval closed-loop adaptive transcranial photobiomodulation stimulation on a person, according to an embodiment of the present invention.

FIG. 4 is a graph showing the dose response curve of photobiomodulation.

FIG. 5 is a graph demonstrating peak Alpha frequency during adaptive closed loop transcranial photobiomodulation according to an embodiment of the present invention.

FIG. 6 is a graph demonstrating peak Alpha frequency without transcranial photobiomodulation.

### DETAILED DESCRIPTION OF THE INVENTION

With reference now to specific embodiments and the accompanying drawings, a detailed description of the invention is provided.

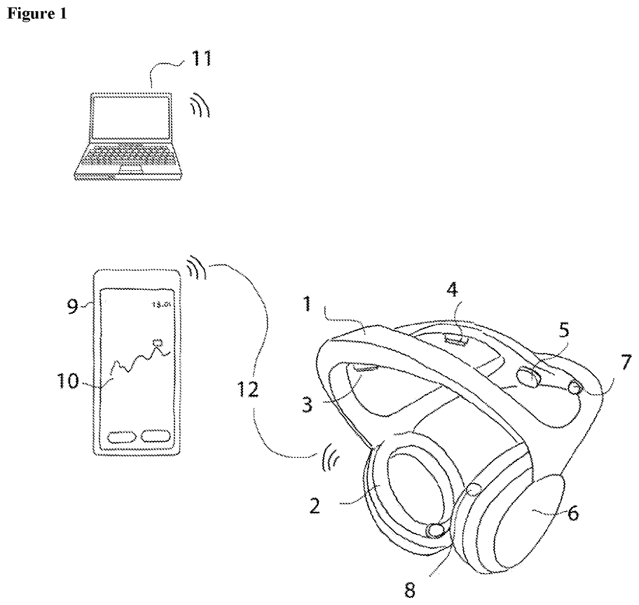

In one aspect, the present invention provides EEG sensors and Photobiomodulation (PBM) LEDs 7 in a head mounted device 1 with headphones 2 and 6 illustrated in FIG. 1. In the embodiment illustrated in FIG. 1, the headphones of the present invention combine EEG (electroencephalography) sensors 3, 4 and 5 for EEG measurement and phoplethysmography (PPG) sensor 8 for heart rate and heart rate variability (HRV) measurement in a wearable head mounted device with headphones. A PPG sensor 8 may be incorporated inside an over-ear headphone design which reduces ambient noise allowing for increased accuracy. The present invention provides a wearable head mounted headphone set 1 with embedded biometric sensors that collect physiological signals from the user. The device includes Bluetooth (wireless) audio and data transmission 12 which may be used to connect the device 1 and 2 to control unit which may be a smartphone or mobile device 9, with graphic touchscreen display 10, and said control unit 9 has wireless wi-fi connection with remotely located master control unit which may be a computer 11. The device 1 may also include a rechargeable battery, speakers, microphone.

The present invention utilizes various Photobiomodulation LEDs located at FZ, F3, F4, CZ, PZ, P3, and P4 according to the international 10-20 placement system. The present invention further utilizes EEG recorded from locations which include Fz, Cz, and Pz according to the international 10-20 placement system. In other embodiments additional or alternative LED and EEG sensor placements may be utilized.

In this method, a targeted brainwave pattern is selected for a Photobiomodulation session. In one embodiment the person selects the desired pattern based on their desired outcomes. In another embodiment, a technician assisting the person may select the target pattern, while in yet another embodiment the system automatically selects or suggests the targeted brainwave pattern based on the person's current biometric readings. Wherein the selected pattern represents one or more target frequencies and one or more target locations. For example a pattern could include 10 Hz at locations Pz, P3, and P4, and 40 Hz at locations FZ, F3, F4, CZ, PZ, P3, and P4. Targeted brainwave patterns may correspond to target states, for example, calm or concentration or meditation.

The person next places the wearable device with EEG sensors and tPBM LEDs on their head. The person uses the control unit to start the session. In one embodiment the device may detect that it is on the person's head and automatically start the session. The device wirelessly transmits biological signal data to the control units. Wherein said biological signal data includes EEG signal data from one or more locations on the person's brain. The control unit applies various signal processing techniques to the biological signal data. Signal processing may include various techniques known to those skilled in the art, including noise filters (i.e. lowpass, highpass, etc.) and analysis techniques (i.e. Fourier transform, Wavelet analysis, etc.). The processed data is used to establish baseline levels for the person including but not limited to average band power and peak band frequency. Wherein brainwave bands include delta, theta, alpha, alpha-theta, low beta, mid meta, high beta, and gamma.

Next the control unit determines a light pulsing pattern for each light location based on the baseline biometric levels and the targeted brainwave patterns and the device applies the pulsing pattern to the LEDs. In this method said light pulsing pattern consists of the following:
1. One or more LED locations.
2. LED duty cycle.
3. LED power output.
4. Light pulse frequency, wherein the light is pulsed on and off at the given frequency.
5. Light pulse duration, wherein the light is pulsed at the specified frequency for the given duration.
6. Light pulse gap duration, wherein the light pulses are paused following the light pulse duration.
7. Repeat duration, wherein the light pulse and gap sequence is repeated for the specified duration.
8. Pause interval, wherein the following the repeated duration, the pattern is paused for a given period of time.

The control unit and device may adjust the energy dose output over time by altering the LED power output, LED duty cycle, and by utilizing light pulse gaps, and pause intervals. The light pulse gap duration and pause interval provide an important advantage by allowing the person's brain time to convert the absorbed energy and stabilize within the individual's dose absorption limit. This further allows the use of higher energy LEDs which penetrate the biological tissue further. Finally this enables control of the dose over time without altering the duty cycle. As the combination of LED pulse frequency and duty cycle impact the exposure time for cells during each individual light pulse.

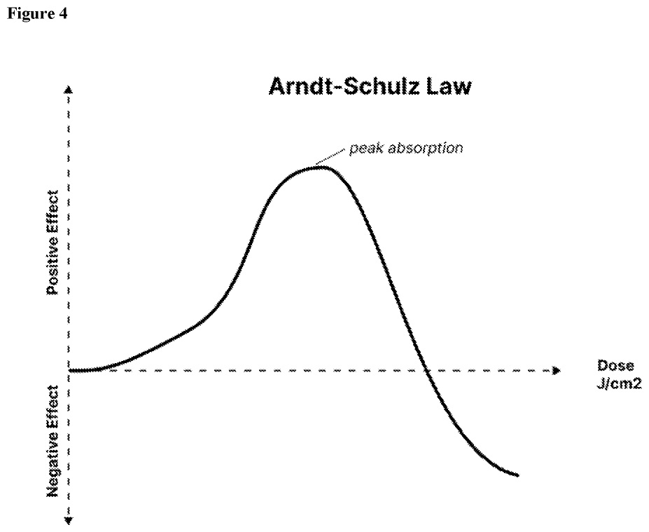

The device and control unit continuously assess the person's biometric signals utilizing various signal analysis and classification techniques. In one embodiment, the control unit monitors the person's EEG power and dose-response curve (FIG. 4). Wherein the control unit maps the person's energy absorption to the dose-response curve based on their EEG power level trend and slope. Where the slope of the person's EEG power increases and decreases in relation to the dose-response curve.

In another embodiment peak alpha frequency is recorded during the baseline period. In this embodiment, the control unit maps the person's energy absorption to the dose-response curve based on their peak alpha frequency trend and slope. Whereas increases to the peak alpha frequency are mapped to the dose-response curve and correspond to continued absorption of the tPBM stimulation and decreases indicate the end of the peak dose-response curve. As the slope of the person's peak alpha frequency changes.

In yet another embodiment of the present invention one or more other biometric indicators may be used to map and measure the dose-response curve for the tPBM stimulation including heart rate (HR), heart rate variability (HRV), pulse volume, respiratory rate, galvanic skin response (GSR), EEG synchronization, EEG amplitude, relative EEG power, and total EEG power.

In the preferred embodiment, the control unit adapts the light pulsing pattern for each light location based on the dose-response curve, targeted brainwave patterns, EEG power, peak band frequencies, baseline biometric levels and changes in the subsequent biometric signals. The system repeats this process for the duration of the Photobiomodulation session. The control unit increases and decreases the tPBM dose over time based on the detected dose-response curve based on the person's mapped biometric levels. The control unit further determines the person has reached the top of the dose-response curve (FIG. 4) as indicated by the biometric levels flattening out. In this embodiment the control unit stops the tPBM session when the top of the dose-response curve is reached.

In an alternative embodiment the device and control unit may be the same physical device. In another embodiment, the control unit transmits the raw signal data to a central master control unit for signal processing. The master control unit may be a centralized server where said adaptation is based on multiple Photobiomodulation sessions. Wherein the system learns to adapt to the individual's optimal dose-response curve over time.

In one embodiment said invention may include 1 or more additional biometric sensors such as body temperature, heart rate, heart rate variability, breathing rate, blood oxygen level (SPO2), respiratory rate, and blood pressure. These additional sensors may be used by the system to further inform the adaptation of the light stimulation intervals.

In another embodiment said light pulsing pattern may include a sequence light pulse frequencies, gaps, and repeat durations. In yet another embodiment all the photobiomodulation lights operate using the same light patterns in unison. In a different embodiment, each photobiomodulation light can be operated independently or in a group with a given light pattern.

In yet another embodiment the system may utilize a history of biometric data collected over time in addition to current biometric signals in order to establish a person's baseline levels. Further the system may learn overtime how the individual responds to changes in light stimulation patterns and incorporate these learning in real-time as it adapts the light patterns.

In yet another variation, the system may include a server and database, wherein learnings from many different user's sessions are used to adapt the light pulsing patterns. In this version the system may include a machine learning algorithm to determine how to adapt the light patterns.

Reference is now made to various working examples and the comparison of the inventive methods with conventional approaches.

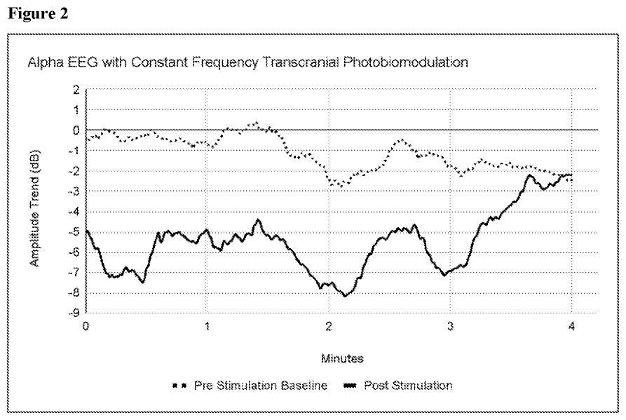

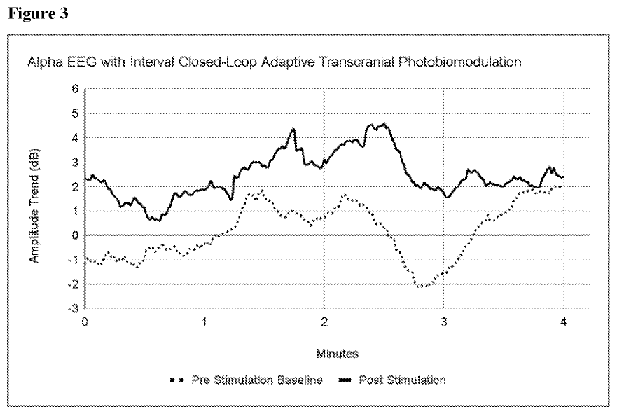

With reference to FIG. 2, this chart demonstrates a significant drop in EEG amplitude using said stimulation method. Data was collected in the 8-12 Hz range (Alpha Band) at the PZ location on the head based on the 10-20 placement system. Wherein an initial 60 second baseline was used to establish the decibel range using a log base 10 scale. After which 4 minutes of pre-stimulation data was collected, followed by 10 minutes of constant frequency stimulation using a frequency of 10 Hz, and then 4 minutes of post stimulation data was collected. FIG. 2 may be directly compared to FIG. 3.

With reference to FIG. 3, this chart demonstrates a significant increase in EEG amplitude using said stimulation method. Data was collected in the 8-12 Hz range (Alpha Band) at the PZ location on the head based on the 10-20 placement system. Wherein an initial 60 second baseline was used to establish the decibel range using a log base 10 scale. After which 4 minutes of pre-stimulation data was collected, followed by 10 minutes of interval closed-loop adaptive stimulation using a light pulse frequency of 10 Hz, and then 4 minutes of post stimulation data was collected. FIG. 3 may be directly compared to FIG. 2.

As discussed already herein, FIG. 4 shows the dose response curve for photobiomodulation. Accordingly, the present inventive methods attempt to stop adaptive transcranial photobiomodulation at or near peak absorption through the use of closed-loop techniques.

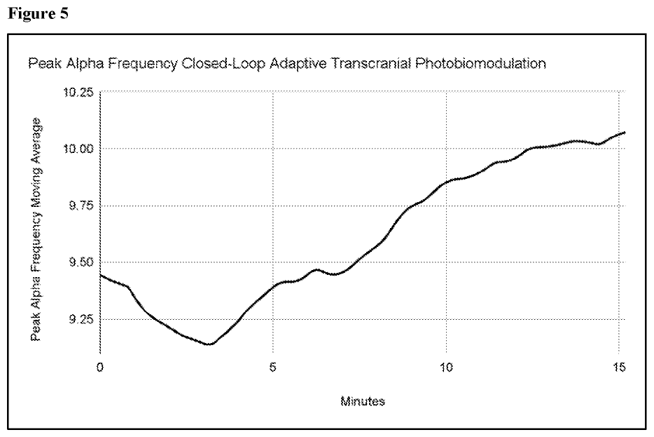

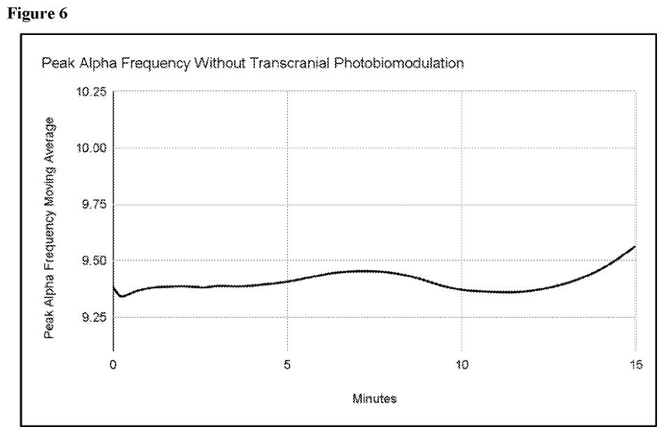

FIGS. 5 and 6 demonstrate the utility and effectiveness of adaptive closed-loop transcranial photobiomodulation according to the inventive methods disclosed herein. As can be seen from examination of these graphs, peak alpha frequency is greatly enhanced through the use of adaptive closed loop techniques. Peak alpha frequency is a research-back correlate to cognitive performance.

The disclosure provided herein is intended to provide exemplary embodiments of the claimed invention, but is not intended to be exclusive or exhaustive. One of skill in the art will understand that variations on the claimed devices and methods are possible without departing from the scope of the claimed invention.

What is claimed is:

1. A method for adaptive transcranial photobiomodulation comprising:
measuring biometric signals of an individual using biometric sensors;
processing the biometric signals;
adapting photobiomodulation based upon the processed biometric signals;
providing photobiomodulation to the individual; and
creating a biofeedback loop wherein the photobiomodulation is adapted to bring the biometric signals of the individual toward a target state.

2. The method of claim 1, wherein the biometric sensors are one or more of EEG, PPG sensors, EKG sensors and galvanic skin response sensors.

3. The method of claim 2, wherein the biometric signals are the measurement of one or more electrical and optical signals that change based on activity from the brain, the heart, blood and the skin.

4. The method of claim 3, wherein the step of measuring the biometric signals comprises determining the baseline biometric signals of the individual.

5. The method of claim 4, wherein the baseline biometric signals may be one or more of band power, peak band frequency, galvanic skin response, blood oxygen levels, pulse volume, heart rate and heart rate variability.

6. The method of any of the preceding claims, further comprising provision of a head mounted wearable device to the individual comprising the biometric sensors, at least one source for the photobiomodulation light pulses and a control unit.

7. The method of claim 6, wherein the target state is set by user or technician input to the control unit, or is selected independently by the control unit.

8. The method of any of the preceding claims wherein the adaptation of the photobiomodulation to bring the biometric signals toward the target state is accomplished by modulating one or more attributes of the PBM and assessing the effect on the measured biometric signals.

9. The method of claim 8, wherein the modulated attributes of the PBM may include one or more of LED location, LED duty cycle, LED power, pulse frequency, pulse duration, pulse gap duration, repeat duration and pause interval.

10. The method of claim 8 or 9 wherein the adaptation of the photobiomodulation is accomplished to bring EEG band power or peak band frequency toward a target state through reference to changes in baseline metrics and creation of a feedback loop.

11. The method of claim 8 or 9 wherein the adaptation of the photobiomodulation is accomplished to bring brain or heart band power, peak band frequency, galvanic skin response, blood oxygen levels, pulse volume, heart rate or heart rate variability toward a target state through reference to changes in baseline metrics and creation of a feedback loop.

12. The method of any of claims 6 through 11, wherein the wearable device further comprises a wireless communication modality allowing for control of the wearable device by a smartphone or other wireless-enabled device.
